(19) United States
(12) Plant Patent Application Publication
(10) Pub. No.: US 2012/0096612 P1
(43) Pub. Date: Apr. 19, 2012

(54) Chinese Dogwood Tree Named 'Losely'

(50) ;

(76) Inventor: Bryan Champion, Perry, OH (US)

(21) Appl. No.: 12/925,104

(22) Filed: Oct. 14, 2010

(51) Int. Cl. A01H 5/00

(52) U.S. Cl. PLT/220

(57) ABSTRACT

A Chinese dogwood named ‘Losely’, a rounded shrubby tree distinguished by the new leaf growth turning bright yellow in mid summer.

### LATIN NAME OF THE GENUS AND SPECIES INCLUDING THE VARIETY DENOMINATION OF THE PLANT CLAIMED

The plant claimed relates to a new and distinct variety of Chinese dogwood, botanically known as Cornus kousa var. chinensis, and known by the cultivar name ‘Losely’.

This new cultivar was selected out of a crop of seedlings originating from uncontrolled pollination, growing at Herman Losely & Son, Inc. in Perry, Ohio. This new cultivar was noted as distinctive because of the bright yellow color on the leaves in mid summer. As summer progresses, the yellow foliage will become flushed with red.

The ‘Losely’ variety is the only variant of Chinese dogwood of which I am aware that displays these foliage characteristics. The following selections of Chinese dogwoods are offered for comparison: Gold Star (unpatented), with gold variegation, but it develops a yellow central leaf blotch that is present the entire growing season; Temple Jewel (unpatented) has a faint yellow central blotch in the spring, disappearing by summer when the leaves are mature; Madison (PP 16129) has gold foliage with red blotching appearing in summer, coloration not consistent from year to year ranging from almost no color to some color.

The original ‘Losely’ cultivar was lost in a flood in 2006. Prior to the loss of the parent plant, the ‘Losely’ cultivar had been asexually reproduced. The oldest of the ‘Losely’ cultivar is approximately 6 years old and is approximately 2.4 meters tall and 2 meters wide. Branching starts at approximately 20 cm above ground level. There are 3 main branches. The largest branch is approximately 4 cm in diameter, the next largest is approximately 3 cm in diameter and the smallest is approximately 2.5 cm in diameter.

The ‘Losely’ cultivar has been asexually reproduced at Herman Losely & Son, Inc. in Perry, Ohio, by means of grafting. All plants propagated by this method have displayed the same unique characteristics of the original plant.

### BRIEF DESCRIPTION OF THE DRAWINGS

FIG. 1 is a colored photograph illustrating the overall appearance of the ‘Losely’ cultivar in the summer.

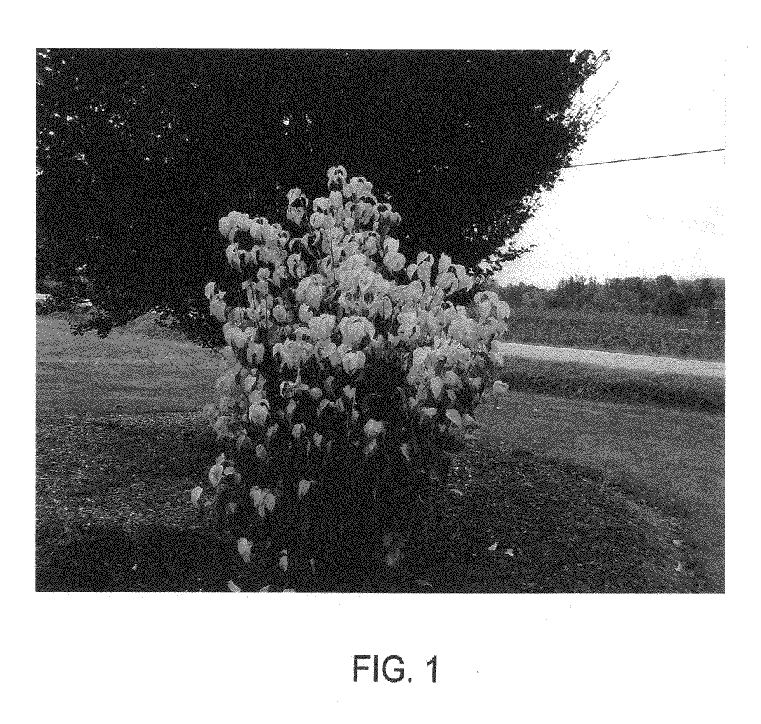

FIG. 2 is a colored photograph illustrating the prolonged yellow coloration on the leaves of ‘Losely’ in the summer.

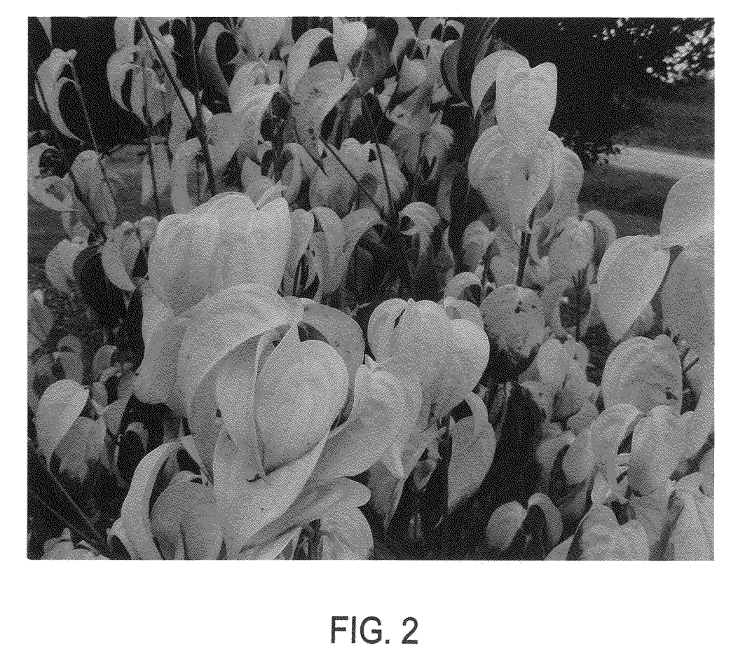

FIG. 3 is a colored photograph illustrating the coloration of the current season's growth found on the inner part of ‘Losely’ in the summer. This coloration may range from green foliage to leaves with yellow advancing from the leaf petiole and the leaf base down the leaf blade to varying degrees.

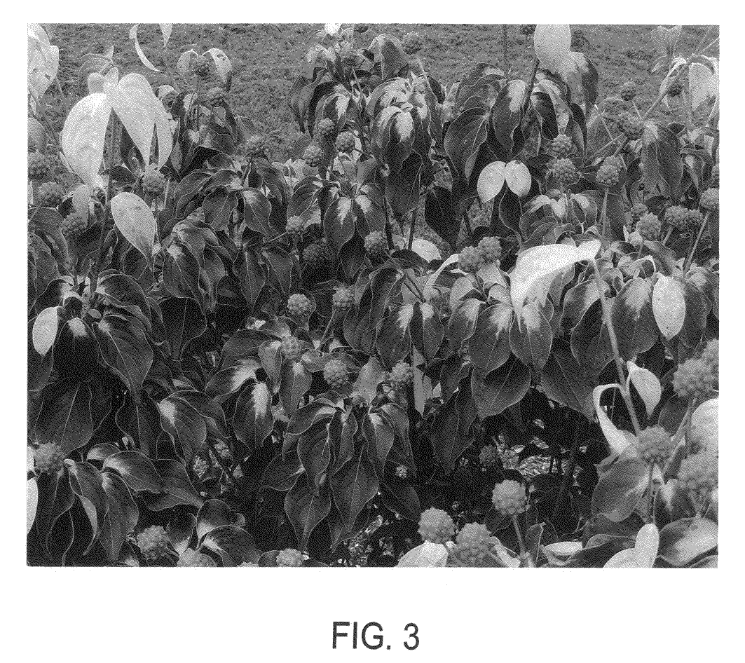

All of the drawing figures show the colors as truly as is reasonably possibly to obtain in colored reproductions of this type.

The following is a detailed description of my new ‘Losely’ cultivar. In all cases, where color is different from the typical and is considered a distinguishing feature of this variety, reference is made to specific colors on the R.H.S. Colour Chart (1995 ed.) published by The Royal Horticultural Society, London, England. Here follows a detailed description of the characteristics of this cultivar, as displayed by the specimens grown at Herman Losely & Son, Inc. in Perry, Ohio. Coloration of leaves and bark may be variable, due to conditions of nutrition, stress, age of plant, location on plant and the presence/absence of sun/shade. Plant part comparisons have been made using samples taken from a mature plant where growth rates and characteristics are considered typical.

### BOTANICAL DESCRIPTION

Parentage: Unknown — selected out of a crop of seedlings originating from uncontrolled pollination, growing at Herman Losely & Son, Inc. in Perry, Ohio.
Hardiness: Hardy in USDA Hardiness Zone 4b (−25 degree F.)
Growth rate: Moderate, more rapid in youth.
Form/size: A shrubby tree 6-10 meters in height and spread at maturity. Rounded as a young plant, maintaining that shape, with a layered, horizontal branching habit. A wide range of factors, especially location, age and if the tree is multi-stem or single stem, influences sizes of stems and branches. It is not possible to make a predictable correlation between the diameter of a primary stem and any branch arising from it.
Stems: Young stems initially smooth gray-green with whitish lenticels present on all bark areas diminishing somewhat on more mature surfaces. Bark on the lower trunk is of the color gray-green (Grayed-green Group 197-A).
Branches: New shoots green and purple turning to gray-orange over time. One and two year twigs are Grayed-Orange 165A. The final 2.5-10 cm of the twig, exclusive of terminal buds may be red-purple (Red-Purple Group 58A) on the upper side and green (Green Group 143C) on the lower side.
Leaves: Deciduous, elliptic-ovate with acuminate tips and cuneate bases 5-12 cm long and 3.5-7.5 cm wide. Leaf stalks are 5-10 mm long. Leaf blades glabrous on top, glabrous underneath, lacking tufts of hairs in leaf axils below. Newly emerging leaves in the spring exhibit yellow-green (Yellow-Green Group 144-B), darkening to green (Green Group 139-A). Many leaves of the current year's growth flush become yellow (Yellow Group 13-B). Coloration begins in late July to August in Perry, Ohio (41.81 degrees North Latitude by 81.13 degrees West Longitude). Overall, current season's growth found on the inner part of the plant may range from green foliage to leaves with yellow advancing from the leaf petiole and the leaf base down the leaf blade to varying degrees. The more vigorous new growth found on the outside canopy exhibits mainly solid yellow leaves. Some leaves will also develop red coloration (Red Group 46-B) over the yellow that appears in the areas of the leaf blade between the veins. The red coloration remains until overtaken by the typical red and orange fall color of the species. The coloration of the foliage appears year after year regardless of weather conditions.
Buds: Buds are two types — Globose, tapering flower buds averaging 7 mm in length by 5 mm in width at base, color gray-brown (Gray-Brown Group 199-A) and sharply tapered vegetative buds averaging 4 mm in length by 2 mm in width at base, color brown (Brown Group 200-B).
Flowers: True flowers are greenish and inconspicuous found in a rounded, central umbel approximately 1 cm wide and 1 cm high. These flowers are surrounded by four partially overlapping tapered creamy white bracts, forming a 4 pointed symmetrical star characterizing each individual inflorescense. Mature bracts most closely resemble Green-White Group 157-A. Bract average 4.5-5 cm long and 3.5-4 cm wide, thereby producing an inflorescense with an overall width of 9-10.5 cm. Flowering commences last week of May to first week of June in Perry, Ohio lasting about six weeks. Flowers of this cultivar are borne more on the inside of the plant and are not overly abundant.
Fruits: The aggregate fruits as for the species are pinkish red, globose and approximately 3 cm in diameter. They are initially borne upright on a 5-6 cm long stalk, later becoming pendulous.

I claim:

1. A Chinese dogwood named ‘Losely’, as described and illustrated, a shrubbery tree distinguished by the bright yellow coloration on the leaves in mid summer.
